(19) United States
(12) Patent Application Publication
(10) Pub. No.: US 2025/0319094 A1
(43) Pub. Date: Oct. 16, 2025

(54) COMPOSITION FOR PREVENTING OR TREATING ATOPIC DERMATITIS

(71) Applicant: Yonsei University BioHealth Technology Holdings, Inc., Seoul (KR)

(72) Inventors: Chang Ook Park, Seoul (KR); Ryeo Won Kim, Seoul (KR); Su Min Kim, Seoul (KR); Kelun Zhang, Seoul (KR); Hye Li Kim, Gyeonggi-Do (KR)

(21) Appl. No.: 18/868,966

(22) PCT Filed: May 25, 2023
(86) PCT No.: PCT/KR2023/007138

§ 371 Date: Nov. 25, 2024

(30) Foreign Application Priority Data
May 26, 2022 (KR) 10-2022-0064531

(51) Int. Cl. A61K 31/5377; A61P 17/00; A61P 37/06; G01N 33/50

(52) U.S. Cl. CPC A61K 31/5377; A61P 17/00; A61P 37/06; G01N 33/5023; G01N 2333/912

(57) ABSTRACT

The present invention relates to a composition for preventing or treating inflammatory or autoimmune skin diseases. The composition of the present invention is able to efficiently ameliorate skin tissue damage caused by inflammation while having a minimized impact on systemic immune activity by specifically inhibiting the activity of skin-specific T cells, thereby addressing the long-term or short-term local side effects of existing treatments for atopic dermatitis.

### TECHNICAL FIELD

The present invention relates to a method of treating inflammatory and autoimmune skin diseases, including atopic dermatitis, using a compound that selectively inhibits skin-specific T cells in atopic dermatitis skin tissue.

### BACKGROUND ART

Atopic dermatitis (AD) is a chronically relapsing, incurable skin disease with a complex pathogenesis, which is primarily caused by the induction of a hypersensitivity response of the immune system by external stimuli. Currently, steroids or immunosuppressive drugs such as cyclosporine, azathioprine, and mycophenolate mofetil (MMF) are mainly used to treat atopic dermatitis, but their effects are transient or not suitable for long-term administration. Topical steroids have the most significant effects as they have a broad spectrum of effects and target a variety of immune molecules in addition to type 2 inflammation, but they pose serious problems when used continuously. For example, infants and young children have a large body surface area relative to their body weight, which can lead to systemic absorption and side effects such as adrenal suppression, and the elderly with thin skin are at high risk of side effects due to excessive absorption of steroid drugs.

Topical calcineurin inhibitors (TCIs), which were developed to overcome the side effects of long-term application of steroidal drugs, are currently widely used, but they also suffer from side effects such as burning and itching. One of the important pathogenetic mechanisms of atopic dermatitis is the infiltration of T cells into the lesions, which leads to impaired skin barrier function and immune system dysregulation. The main problem with existing immunosuppressive drugs is that, despite the fact that atopic dermatitis is a tissue-specific disease that occurs in the skin, most of them have been developed to control immune and inflammatory responses through a systemic approach, specifically targeting the blood. The search for new therapeutic candidates for atopic dermatitis requires a novel therapeutic approach that targets skin-specific T cells, but this is not being done at all globally.

Therefore, the inventors have discovered compounds that selectively act on skin-specific T cells without systemic immunosuppression, while also improving short-term skin irritation symptoms, thereby have completed the present invention.

### DISCLOSURE

### Technical Problem

An object of the present invention is to provide pharmaceutical compositions for the prevention or treatment of inflammatory or autoimmune skin diseases.

Another object of the present invention is to provide a cosmetic composition for the prevention or amelioration of inflammatory or autoimmune skin diseases.

Still another object of the present invention is to provide a transdermal dosage form or topical skin application for the prevention or treatment of inflammatory or autoimmune skin diseases.

However, the technical challenges of the present invention are not limited to those mentioned above, and other challenges not mentioned will become apparent to one of ordinary skill in the art from the following description.

### Technical Solution

One embodiment of the present invention provides a pharmaceutical composition for the prevention or treatment of inflammatory or autoimmune skin diseases.

The pharmaceutical compositions of the present invention comprise a compound of the following Formula 1 or a pharmaceutically acceptable salt thereof as an active ingredient:

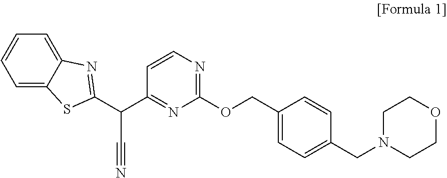 [SMILES: N#CC(c1ccnc(OCc2ccc(CN3CCOCC3)cc2)n1)c1nc2ccccc2s1]

The compound represented by Formula 1 in the present invention has the CAS number 848344-36-5, and its synonyms are Bentamapimod; AS-602801; α-[2-[[4-(4-morpholinylmethyl)phenyl]methoxy]-4-pyrimidinyl]-2-benzothiazoleacetonitrile; and others. Bentamapimod is an orally active, selective JNK inhibitor (c-Jun N-terminal kinases inhibitor) that blocks T cell proliferation and induces apoptosis. It is currently in clinical trials for the treatment of endometriosis, among other conditions, and is being investigated as a drug candidate for prostate cancer, but there are no reports on its ability to modulate the activity of skin-specific T cells, and its effectiveness in treating autoimmune skin diseases, including atopic dermatitis.

In the present invention, a pharmaceutically acceptable salt is a salt that is generally considered by those skilled in the art to be suitable for medical application (e.g., because such a salt is not harmful to a subject that may be treated with the salt), or a salt that produces acceptable side effects within the respective treatment. Generally, said pharmaceutically acceptable salts are those considered acceptable by regulatory authorities such as the U.S. Food and Drug Administration (FDA), the European Medicines Agency (EMA), or the Pharmaceuticals and Medical Devices Agency (PMDA) of the Japanese Ministry of Health, Labor and Welfare. However, the invention also encompasses, in principle, salts of compounds according to the invention that are not pharmaceutically acceptable in themselves, for example, as intermediates in the preparation of compounds according to the invention or physiologically active derivatives thereof, or as intermediates in the preparation of pharmaceutically acceptable salts of compounds according to the invention or physiologically active derivatives thereof. Said salts include water-insoluble salts and, in particular, water-soluble salts.

In each case, a person skilled in the art can readily determine whether a particular compound according to the invention or a physiologically active derivative thereof is capable of forming a salt, i.e., whether the compound according to the invention or a physiologically active derivative thereof has a charged group, such as, for example, an amino group, a carboxylic acid group, or the like.

Exemplary salts of the compounds of the invention are acid adduct salts or salts with bases, in particular pharmaceutically acceptable inorganic and organic acid adduct salts and salts with bases conventionally used in pharmacy, which are water-insoluble or particularly water-soluble acid adduct salts. Depending on the substituent groups of the compounds of the invention, salts with bases may also be suitable. Acid addition salts can be formed, for example, by mixing a solution of a compound of the invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Similarly, pharmaceutically acceptable base addition salts can include alkali metal salts (e.g., sodium or potassium salts); alkaline earth metal salts (e.g., calcium or magnesium salts); and salts formed with suitable organic ligands (e.g., ammonium, quaternary ammonium, and amine cations formed using opposing anions such as halides, hydroxides, carboxylates, sulfates, phosphates, nitrates, alkyl sulfonates, and aryl sulfonates). Exemplary pharmaceutically acceptable salts include acetate, adipate, alginate, arginate, ascorbate, aspartate, benzenesulfonate, benzoate, bicarbonate, bisulfate, bisulfate, bitartrate, borate, bromide, butyrate, calcium edetate, camphorate, camphorsulfonate, campsylate, carbonate, chloride, citrate, digluconate, dihydrochloride, dodecyl sulfate, edetate, edicylate, ethanesulfonate, formate, fumarate, galactate, galacturonate, gluconate, glutamate, glycerophosphate, hemisulfate, heptanoate, hexanoate, hexylresorcinate, hydrobromide, hydrochloride, Hydroiodide, 2-hydroxy-ethanesulfonate, hydroxynaphthoate, iodide, isobutyrate, isothionate, isothionate, lactate, laurate, lauryl sulfate, malate, maleate, malonate, mandelate, methanesulfonate (mesylate), methyl sulfate, 2-naphthalenesulfonate, nicotinate, nitrate, oleate, oxalate, palmitate, pantothenate, pectinate, persulfate, 3-phenylpropionate, phosphate/diphosphate, phthalate, picrate, pivalate, polygalacturonate, propionate, salicylate, stearate, sulfate, succinate, succinate, tannate, tartrate, tosylate, undecanoate, valerate, and the like.

Salts which are not pharmaceutically acceptable in the invention and which may be obtained, for example, as process products during the manufacture of compounds according to the invention on an industrial scale are also included in the invention and, if desired, may be converted to pharmaceutically acceptable salts by methods known to those skilled in the art.

In the present invention, the term “inflammatory or autoimmune skin disease” refers to a disease in which skin tissue is damaged and the inherent biological functions of skin tissue, including barrier function, are reduced, due to excessive or unwanted immune response or inflammation caused thereby. Said inflammatory or autoimmune skin disease may be at least one selected from the group consisting of atopic dermatitis, eczema, psoriasis, allergies, erythema multiforme, erythema nodosum, and gangrenous pyoderma, and more specifically may be atopic dermatitis, but is not limited to.

As used in the present invention, atopic dermatitis refers to a chronic allergic inflammatory skin reaction that occurs on the skin and is common in children and can persist into adulthood. Atopic dermatitis is more likely to occur if there are other allergic diseases such as asthma or allergic rhinitis in the family, and can be caused by allergens such as food or house dust mites. Atopic dermatitis is also known to be exacerbated by sudden changes in indoor temperature and humidity, sweating or saliva, tight or rough clothing, rubbing or scratching the skin, stress, and bacterial infections. In addition, although various medications are currently available for the treatment of atopic dermatitis, steroid medications cause various local side effects, including skin atrophy, dilated skin capillaries, and the formation of hypopigmented patches, and topical calcineurin inhibitor treatments have been developed to avoid causing various long-term local side effects, including skin atrophy, and topical calcineurin inhibitor treatments have been developed to avoid causing various long-term local side effects, including skin atrophy, dilated skin capillaries, and hypopigmented patches, However, short-term skin irritation has emerged as a new problem, making it imperative to develop more effective treatments.

As shown in the following embodiments, the compounds of the present invention can significantly improve skin lesions and suppress skin irritation symptoms in animal models of atopic dermatitis, as well as reduce the number and activity of skin-specific T cells present in skin tissues such as epidermis and dermis, thereby exerting a lesion-specific and focused immunocontrol effect compared to conventional immunosuppressive agents that cause systemic immunosuppression. Thus, in refractory autoimmune skin diseases with complex pathogenesis, specifically atopic dermatitis, controlling skin tissue-specific inflammatory and immune responses can efficiently improve the symptoms of skin lesions.

As used herein, “preventing” means inhibiting the occurrence of a disorder or disease in a subject who has never been diagnosed as having the disorder or disease, but is likely to suffer from such disorder or disease.

As used herein, “treating” means (a) inhibiting the progress of a disorder, disease or symptom; (b) alleviating the disorder, disease or symptom; or (c) eliminating the disorder, disease or symptom. When the composition of the present invention is administered to a subject, it functions to inhibit skin-specific T cells present in skin tissue, thereby inhibiting the progress of symptoms caused by excessive immune and inflammatory responses in skin tissue, or eliminating or alleviating the symptoms. Thus, the composition of the present invention may serve as a therapeutic composition for the disease alone, or may be administered in combination with other pharmacological ingredients and applied as a therapeutic aid for the disease.

As used herein, “treating” or “administering” refers to directly administering a therapeutically effective amount of a composition of the invention to a subject so that the same amount is formed in the subject's body.

As used herein, “therapeutically effective amount” means a content of the composition that contains a pharmaceutical ingredient in the composition in an amount sufficient to provide a therapeutic or prophylactic effect to an individual to whom the pharmaceutical composition of the invention is to be administered, and includes “prophylactic effective amount”.

As used herein, the term “subject” may include a human, mouse, rat, guinea pig, dog, cat, horse, cow, pig, monkey, chimpanzee, baboon, or rhesus monkey, and more specifically may be a human, but is not limited to.

In the present invention, the composition can inhibit the proliferation of T cells, more specifically, the composition can inhibit proliferation by reducing the number or activity of skin-specific T cells.

When the compositions of the invention are formulated as pharmaceutical compositions, the pharmaceutical compositions of the invention may include pharmaceutically acceptable carriers. Pharmaceutically acceptable carriers for inclusion in the pharmaceutical compositions of the present invention are those customarily utilized in pharmaceutical formulations, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oils. In addition to the above ingredients, the pharmaceutical compositions of the present invention may further comprise lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19th ed., 1995).

The pharmaceutical compositions of the present invention can be administered parenterally, and more specifically, can be administered transdermally, subcutaneously, or topically applied to a skin surface. More specifically, the pharmaceutical compositions of the present invention can be transdermal administration or topical skin applications, but are not limited to.

Suitable dosages of the pharmaceutical compositions of the invention may be prescribed in a variety of ways, depending on factors such as method of formulation, mode of administration, patient age, weight, sex, medical condition, food, time of administration, route of administration, rate of excretion, and response sensitivity. Preferred dosages of the pharmaceutical compositions of the present invention are in the range of 0.001-100 mg/kg in adults.

The pharmaceutical composition of the present invention may be prepared in a unit dose form or prepared to be contained in a multi-dose container by formulating with a pharmaceutically acceptable carrier and/or excipient, according to a method that may be easily carried out by a person skilled in the art.

Other embodiments of the present invention provide cosmetic compositions for the prevention or improvement of inflammatory or autoimmune skin diseases.

The cosmetic composition of the present invention comprises a compound of the following Formula 1 or a cosmetically acceptable salt thereof as an active ingredient:

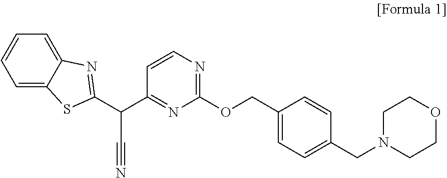 [SMILES: N#CC(c1ccnc(OCc2ccc(CN3CCOCC3)cc2)n1)c1nc2ccccc2s1]

In the compositions for the prevention or treatment of inflammatory or autoimmune skin diseases of the present invention, the description of the compound represented by Formula 1 and the inflammatory or autoimmune skin disease or diseases is redundant to the foregoing and is hereby omitted from further description.

In the present invention, the term “cosmetically acceptable salt” refers to a salt that is a substance in which cations and anions are held together by electrostatic attraction, and a salt in a form that can be used cosmetically, and specific examples of the kind may include examples of the term “pharmaceutically acceptable salt” described above.

In addition to the compounds of Formula 1 or cosmetically acceptable salts thereof as active ingredients, the ingredients included in the cosmetic compositions of the present invention may include ingredients conventionally used in cosmetic compositions, such as, but not limited to, antioxidants, stabilizers, solubilizers, conventional adjuvants such as vitamins, pigments and fragrances, and carriers.

The cosmetic compositions of the present invention can be formulated in any formulation conventionally formulated in the art, for example solutions, suspensions, emulsions, pastes, gels, creams, lotions, powders, soaps, surfactant-containing cleaners, oils, powder foundations, emulsion foundations, wax foundations, and sprays, but not limited to.

If the formulation of the present invention is a paste, cream, or gel, it may comprise, as a carrier ingredient, animal oil, vegetable oil, wax, paraffin, starch, tragacanth, a cellulose derivative, polyethylene glycol, silicone, bentonite, silica, talc, zinc oxide, or the like.

If the formulation of the present invention is a powder or spray formulation, it may comprise, as a carrier ingredient, lactose, talc, silica, aluminum hydroxide, calcium silicate, or polyamide powder. Particularly, if the formulation is a spray formulation, it may further comprise a propellant, such as chlorofluorohydrocarbon, propane/butane, or dimethyl ether.

If the formulation of the present invention is a solution or emulsion, it may comprise, as a carrier ingredient, a solvent, a solubilizing agent, or an emulsifying agent, In this case, examples of the carrier ingredient include water, ethanol, isopropanol, ethyl carbonate, ethyl acetate, benzyl alcohol, benzyl benzoate, propylene glycol, 1,3-butyl glycol oil, glycerol fatty ester, polyethylene glycol, or sorbitan fatty acid ester.

If the formulation of the present invention is a suspension, it may comprise, as a carrier ingredient, a liquid diluent, such as water, ethanol, or propylene glycol, a suspending agent, such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like.

If the formulation of the present invention is a surfactant-containing cleanser, it may comprise, as a carrier ingredient, aliphatic alcohol sulfate, aliphatic alcohol ether sulfate, sulfosuccinic monoester, isethionate, an imidazolium derivative, methyl taurate, sarcosinate, fatty acid amide ether sulfate, alkyl amido betaine, aliphatic alcohol, fatty acid glyceride, fatty acid diethanolamide, vegetable oil, a lanoline derivative, ethoxylated glycerol fatty acid ester, or the like.

Still another embodiment of the present invention provides a method of preventing, ameliorating, or treating an inflammatory or autoimmune skin disorder, comprising the step of administering or applying a subject in need thereof a composition for preventing, ameliorating, or treating an inflammatory or autoimmune skin disorder provided herein.

As used herein, the term “subject” refers to a mammal, including mice, livestock, and the like, including humans, that has developed or is suspected of developing an inflammatory or autoimmune skin disease, but includes, without limitation, any subject treatable with the compositions provided herein.

The methods of the present invention may comprise administering a composition provided herein in a pharmaceutically or cosmetically effective amount.

The dosage, schedule, and route of administration of the compositions provided by the present invention can be determined based on the size and condition of the individual and in accordance with standard practice.

The amount administered to an individual in the present invention may vary, for example, depending on the particular formulation of the active ingredient being administered, the route of administration, and the particular type of disease being treated and the degree of progression of the disease. The amount should be sufficient to produce a desired response, such as a therapeutic response to an inflammatory or autoimmune skin condition, without severe toxicity or harmful events. In some embodiments, the amount of the active ingredient is sufficient to reduce the progression of the disease, improve the disease, or cure the disease by at least one of about 10%, 20%, 30%, 40%, 50%, 60%, 70%, 80%, 90%, 95%, or 100% compared to the corresponding disease in the same individual prior to treatment, or compared to the corresponding activity in another untreated individual. The magnitude of the effect can be measured using standard methods such as in vitro assays, cell-based assays, animal models, or human trials.

In the methods of preventing, ameliorating, or treating inflammatory or autoimmune skin diseases of the present invention, the following descriptions of compositions for preventing, ameliorating, or treating inflammatory or autoimmune skin diseases, inflammatory or autoimmune skin diseases, and the like are redundant and will not be described in detail below.

Yet another embodiment of the present invention provides a method for screening compositions for skin-specific T cell inhibition comprising the steps of:
(a) bringing a candidate substance into contact with a biological sample containing cells expressing an c-Jun N-terminal kinases (JNK) protein;
(b) measuring the activity or expression level of the JNK protein in the sample,
wherein, if the activity or expression level of the JNK protein decreased, the candidate substance is determined as the composition for inhibiting skin-specific T cells.

In the present invention, the term “biological sample” refers to any sample containing cells expressing JNK protein, obtained from mammals, including humans, and includes, but is not limited to, tissues, organs, cells, or cell cultures. Further, the biological sample may comprise skin tissue or skin tissue-derived cells. The skin tissue-derived cells may include, but are not limited to, keratinocytes and dermal fibroblasts.

As used herein, “candidate” refers to an unknown substance that is added to a sample comprising cells expressing JNK and utilized in a screening to test whether it affects the activity or expression of JNK at the gene or protein level. The test substance may include, but is not limited to, a compound, nucleotide, peptide, or natural extract. The step of measuring the expression level or activity of JNK in the biological sample treated with the test substance may be performed by various expression level and activity measurement methods known in the art. As a result of the measurement, when the expression level or activity of the JNK decreased, the test substance may be determined as an inhibitor that reduces the number and/or activity of skin-specific T cells present in skin tissue.

As used herein, “decrease in activity or expression” means a decrease in the inherent in vivo function or expression of JNK such that skin-specific T cell activation of JNK identified by the inventors is significantly inhibited, resulting in a measurable improvement in inflammatory damage in skin lesions. A decrease in the activity includes not only a simple decrease in function but also an ultimate decrease in the activity due to a decrease in stability. Specifically, the term “decrease in activity or expression level” may mean a state in which the activity or expression level decreased by at least 20%, more specifically at least 40%, even more specifically at least 60%, compared to that in a control group.

### Advantageous Effects

By specifically inhibiting the activity of skin-specific T cells, the present invention may be useful as a fundamental therapeutic composition that can efficiently ameliorate inflammation-induced skin tissue damage with minimal impact on systemic immune activity and without inducing skin irritation symptoms. Furthermore, the compositions of the present invention can be efficiently delivered to the stratum corneum of the skin to exert lesion-specific and intensive immune control as a topical skin application, while exhibiting little toxicity to normal skin tissue, making them suitable for long-term administration for the treatment of the chronic disease atopic dermatitis.

### BRIEF DESCRIPTION OF DRAWINGS

FIG. 1 shows HDM treatment of flaky tail mice to induce skin lesions of atopic dermatitis in accordance with one embodiment of the present invention.

FIGS. 2A and 2B are schematic diagrams of the JNK signaling cascade according to one embodiment of the present invention, and heatmaps of signaling proteins expressed in the JNK signaling system that have elevated expression in skin-specific T cells present in skin tissue from atopic dermatitis, respectively.

FIG. 3 is a histogram of cell viability (CCK-8) results showing the cytotoxicity of Bentamapimod against normal skin tissue in accordance with one embodiment of the present invention.

FIG. 4 is a diagram modeling a timeline of HDM treatment and drug treatment for a mouse model of atopic dermatitis according to one embodiment of the present invention.

FIG. 5 is a clinical photograph illustrating the inflammatory response and treatment effects of various drugs on a mouse model of atopic dermatitis induced by HDM according to one embodiment of the present invention.

FIG. 6 is a diagram illustrating the change in mouse dermatitis score (Flaky tail Mice dermatitis score) of a mouse model of atopic dermatitis following treatment with various drugs according to one embodiment of the present invention.

FIG. 7 is a diagram of wiping and scratching behavior of Flaky tail mice by drug group according to one embodiment of the present invention.

FIGS. 8a and 8b are graphical representations of the quantification of the number of acute wiping and scratching behaviors by drug group in flaky tail mice according to one embodiment of the present invention (**P<0.05).

FIGS. 9A and 9B are graphs comparing chronic itch behavior before and after application of test drug to Flaky tail mice in accordance with one embodiment of the present invention. Data are expressed as mean±standard deviation (SD) (*P<0.1, **P<0.01).

FIG. 10a is a diagram showing a comparison of T cell fractions in the skin of a mouse model of atopic dermatitis following treatment with a test drug according to one embodiment of the present invention. Data are expressed as mean±standard deviation (SD) (*P<0.1, **P<0.01).

FIG. 10B is a diagram showing a comparison of T cell fractions in skin-draining lymph nodes of a mouse model of atopic dermatitis following treatment with a test drug according to one embodiment of the present invention. Data are expressed as mean±standard deviation (SD) (*P<0.1, **P<0.01).

FIG. 11 shows the results of a comparison of T cell fractions using skin immune cells and skin lesions from a patient with atopic dermatitis following treatment with a test drug according to one embodiment of the present invention. Data are expressed as mean±standard deviation (SD) (*P<0.1, **P<0.01).

### BEST MODE

One embodiment of the present invention relates to a pharmaceutical composition for preventing or treating inflammatory or autoimmune skin diseases comprising a compound represented by Formula 1 below or a pharmaceutically acceptable salt thereof as an active ingredient.

Another embodiment of the invention relates to a cosmetic composition for the preventing or ameliorating inflammatory or autoimmune skin diseases comprising a compound represented by Formula 1 below or a cosmetically acceptable salt thereof as an active ingredient.

Still another embodiment of the present invention relates to a method of preventing, ameliorating, or treating an inflammatory or autoimmune skin disease, comprising the step of administering to a subject in need thereof a composition comprising a compound represented by Formula 1 below, or an acceptable salt thereof as an active ingredient, provided herein.

Yet another embodiment of the present invention relates to a method of screening a composition for inhibiting skin-specific T cells, comprising steps of (a) bringing a candidate substance into contact with a biological sample containing cells expressing an c-Jun N-terminal kinases (JNK) protein; and (b) measuring the activity or expression of JNK proteins in the sample, wherein if the activity or expression of the JNK protein decreased, the candidate substance is determined as the composition for inhibiting skin-specific T cells.

### MODE FOR INVENTION

Hereinafter, the present invention will be described in further detail by examples. It would be obvious to those skilled in the art that these examples are intended to be more concretely illustrative and that the scope of the present invention as set forth in the appended claims is not limited to or by the examples.

### Preparation Example: Preparation of an Atopic Dermatitis Mouse Model

Flaky tail mice exhibiting a clinical and histological phenotype similar to atopic dermatitis were obtained and stabilized for 1 week under laboratory conditions of 23±2° C., 55±5% humidity and 12 h/12 h (light/dark cycle). The backs of the mice were shaved and exfoliated with a 4% diluted sodium dodecyl sulfate solution (SIGMA Lot #BCCD5615) once every 3 days to remove dead skin cells, and House Dust Mite (HDM), a typical trigger/exacerbator of atopic dermatitis, was topically applied to the mice to induce atopic skin lesions. The house dust mite ointment used was Atopic dermatitis challenging ointment from Biostir (Osaka, Japan), and atopic dermatitis-induced mice were prepared as described above (see FIG. 1).

### Example 1: Discovery of a Drug to Treat Atopic Dermatitis

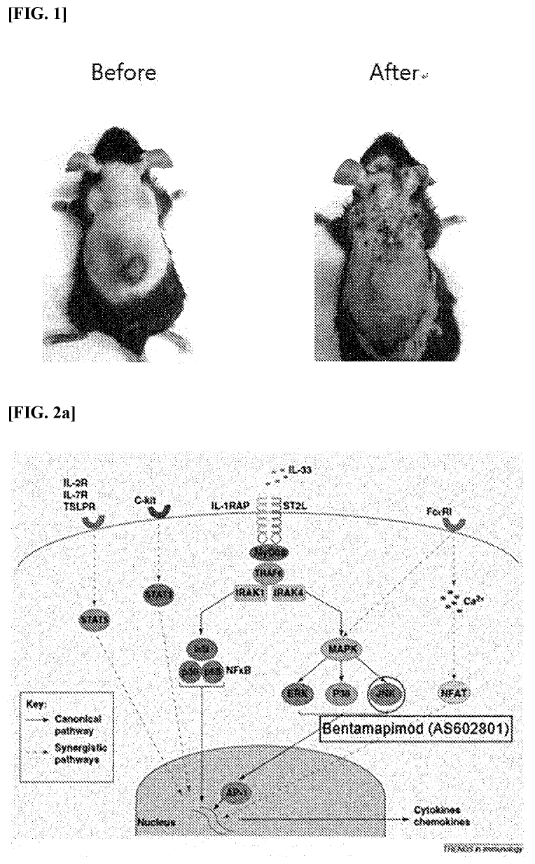

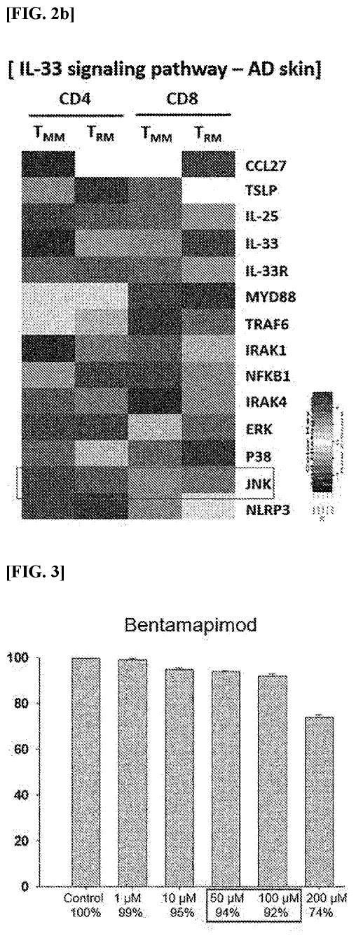

Having identified that T cell activity in atopic dermatitis skin lesions can be suppressed through modulation of the JNK pathway signaling pathway, the inventors sought to screen for skin-specific and stable drugs that inhibit the expression of this pathway. Specifically, we used transcriptomics and bioinformatics tools to identify molecules responsible for signal transduction in the JNK signaling cascade signaling system, which are specifically expressed by skin-specific T cells, as candidate drug targets. Heat maps were created and statistical analyses were performed to identify signaling proteins expressed in the JNK signaling cascades signaling system that are elevated in skin-specific T cells present in atopic dermatitis skin, and the results are shown in FIG. 2a and FIG. 2b.

Experiments have shown that IL-33 induces T hepler type 2 (Th2) responses by activating the Suppression of Tumorigenicity 2 (ST2)/IL-1 receptor accessory protein receptor on various cell types, including mast cells and Th2 cells, Since it is widely believed to play an important role in allergic and atopic diseases, we wanted to screen drugs that target the downstream signaling of IL-33, and identified JNK, a protein whose expression is increased in CD4 T cells compared to CD8 T cells, and showed a significant increase in CD4 TMM (migrated memory T cells) and TRM (tissue-resident memory), and finally selected Bentamapimod among the drugs involved in JNK signaling.

### Example 2: Evaluating the Cytotoxicity of the Drug Bentamapimod

We sought to determine the optimal concentration of the drug Bentamapimod identified above that would not affect the cytotoxicity of the drug. To determine the final concentration of the drug, normal skin tissue was seeded as cells in 2×45 96-well plates using 5% (v/v) RPMI1640 medium (LONZA) and 1% penicillin/streptomycin medium and treated with bentamapimod at concentrations of 1 μM, 10 μM, 50 μM, 100 μM, and 200 μM, respectively, and incubated overnight in a 37° C. incubator. Then, 10 μl of Cell Counting Kit-8 drug (Dojindo Lot. PR863) was seeded into 96-well plates and the absorbance was measured at 540 nm after 2 h. The changes in cell viability are shown in FIG. 3.

The experimental results confirmed that bentamapimod was safe and non-cytotoxic to normal skin tissues in the concentration range of 50 μM and 100 μM, as the cell viability was greater than 90% at 50 μM and 100 μM concentrations.

### Example 3: Validating the Therapeutic Effectiveness of the Drug Bentamapimod

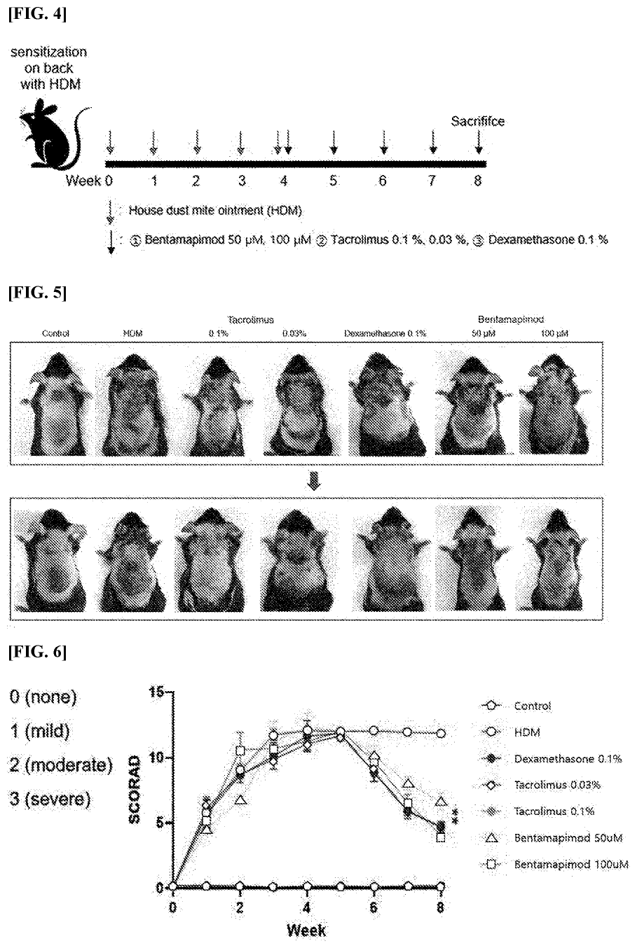

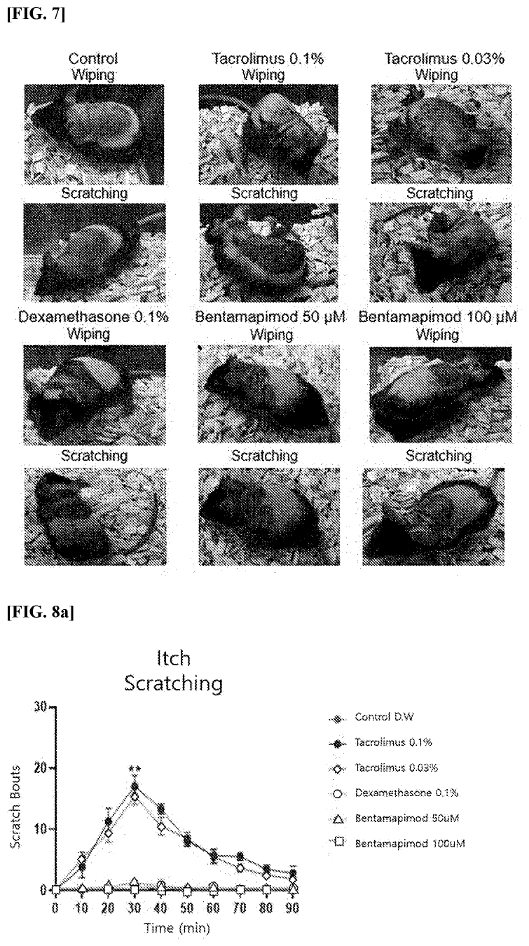

In order to determine whether the bentamapimod drug discovered above has a therapeutic effect on atopic dermatitis, experiments were conducted using the mouse model prepared in the preliminary example and divided into control and experimental groups according to the timeline diagram shown in FIG. 4. The negative control group is a flaky-tailed mouse without HDM treatment, the positive control group is a flaky-tailed mouse treated with dexamethasone, a topical steroidal agent, and tacrolimus, a topical calcineurin inhibitor, among the drugs widely used as atopic dermatitis drugs after HDM treatment, and the experimental group is a flaky-tailed mouse treated with bentamapimod after HDM treatment. Specifically, HDM 100 mg was applied to the backs of mice three times a week for 4 weeks to induce atopic dermatitis, and then the mice were treated with the corresponding drugs by concentration (dexamethasone 0.1%, tacrolimus 0.1% or 0.03%, bentamapimod 50 μM or 100 μM; % concentration is converted to the number of g of dissolved solute in 100 g) in 200 μL on the backs of mice and observed after 4 weeks, and the results are shown in FIG. 5. To further verify the treatment effect, the flaky tail Mice dermatitis score of the atopic dermatitis mouse model according to the drug treatment was calculated by Wilcoxon-signed rank test and shown in FIG. 6. A p-value of less than 0.05 was considered significant.

Referring to FIG. 5, the results showed that treatment with bentamapimod showed a clinically significant treatment effect on atopic dermatitis as compared to conventional drugs, with significant reductions in wounding, dryness, desquamation, erythema, hemorrhage, and edema, especially when compared to the positive control group of 0.1% dexamethasone. Referring to FIG. 6, the results also showed that treatment with the newly discovered bentamapimod drug resulted in atopic dermatitis scores that were equivalent to or better than those of conventional drugs.

### Example 4: Identifying Improved Therapeutic Effects of the Drug Bentamapimod (1)

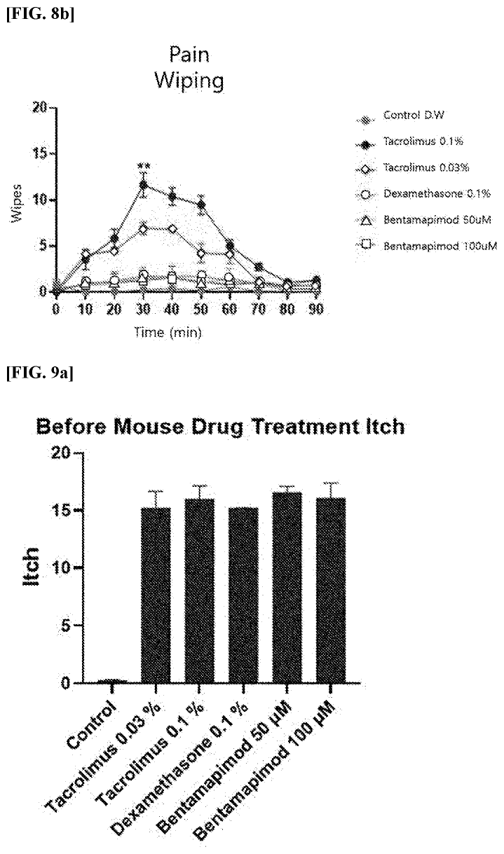

The existing drug dexamethasone is widely used as a treatment for atopic dermatitis, but long-term use causes various local side effects such as skin atrophy, dilated skin capillaries, and hypopigmented spots. Tacrolimus, which was developed to minimize these long-term local side effects, solved the problem of long-term and permanent local side effects of steroid drugs, but short-term skin irritation symptoms are emerging as a new problem, as it is known that about 50% of patients experience burning and skin rash at the beginning of application. Therefore, it is necessary to overcome the skin irritation side effects, and we wanted to check whether the newly discovered drug of bentamapimod can resolve the above side effects while maintaining the effectiveness of the animal drug through behavioral analysis of pain and itching using Flaky Tail mice. Wiping the face with the forelegs was interpreted as a behavioral measure of pain in mice, and scratching with the hind legs was interpreted as a behavioral measure of itching (J Vis Exp. 2019 Sep. 27; (151)). Specifically, 200 μL of tacrolimus 0.1%, 0.03%, and bentamapimod 50 UM and 100 μM were applied to the back of atopic dermatitis-induced flaky tail mice, and the pain behavior (Wiping) and itching behavior (Scratching) were observed for 90 minutes, and the results are shown in FIG. 7. Flaky tail mice were treated with 200 μL of the test agent per group for one week to facilitate comparison, and the number of itches and pain behaviors were quantified and graphed (see FIG. 8a and FIG. 8b).

As a result of the experiment, the number of scratching behaviors was the highest at 0.1% concentration of tacrolimus, a topical calcineurin inhibitors (TCIs) drug, at 17 times in 30 minutes, and wiping behaviors peaked at 30 minutes at 0.1% concentration of tacrolimus. In addition, we found that there was no difference in the number of scratching and wiping behaviors between the bentamapimod 50 μM and 100 μM groups and the control D.W group. Based on the above results, it was confirmed that the bentamapimod drug did not cause local irritation symptoms such as itching and pain.

### Example 5: Identifying Improved Therapeutic Effects of the Drug Bentamapimod (2)

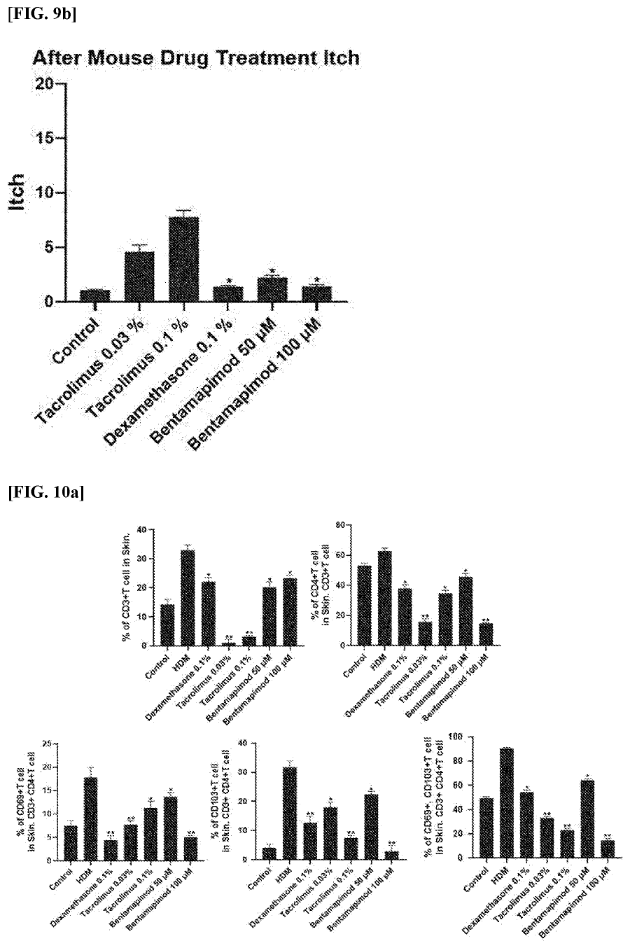

Additional experiments were performed to compare the ITCH behavior before and after application of the dexamethasone, tacrolimus, and bentamapimod agents disclosed above. Specifically, atopic dermatitis was induced by applying 100 mg of HDM ointment to the backs of flaky tail mice for 4 weeks, after which drug treatments were applied 3 times a week for 4 weeks, and the chronic itch behavior before and after application of these test drugs was quantified and graphed (see FIG. 9a and FIG. 9b).

The results showed that scratching behavior was reduced, and the treatment effect was particularly significant in the bentamapimod group. This was comparable to the effect of dexamethasone, suggesting that the newly discovered bentamapimod drug could be utilized as a treatment for atopic dermatitis that overcomes long-term and permanent local side effects while improving short-term skin irritation.

### Example 6: Effects of the Drug Bentamapimod on Skin-Specific T Cells

### 6.1 Identifying Changes in Skin-Specific T Cells in Flaky Tail Mice

To test the effect of the newly discovered bentamapimod drugs on skin-specific T cells, flaky tail mice were induced with atopic skin lesions using HDM three times a week until week 4 of the experiment as described in the Preparations and Examples above, and then treated with each drug at different concentrations for another 4 weeks. To determine changes in skin-specific T cells in each animal, single suspensions were then prepared from spleen, skin, and lymph nodes, and T cells were stained with anti-CD3, anti-CD4, anti-CD8, and anti-CD45 antibodies. Flow cytometric analysis of skin-specific T cells was performed using anti-CD69 and anti-CD103 antibodies to observe changes in the number and distribution of skin-specific T cells in response to drug application.

The results showed that the percentage of CD3+, CD4+ T cells in the skin was significantly reduced in a dose-dependent manner in both the 50 μM and 100 μM groups of bentamapimod, and in particular, the percentage of CD69+ T cells and CD103+ T cells (skin-resident T cells) was significantly reduced by bentamapimod, and the percentage of skin-specific T cells was significantly reduced in the bentamapimod group compared to the control 0.1% dexamethasone group.

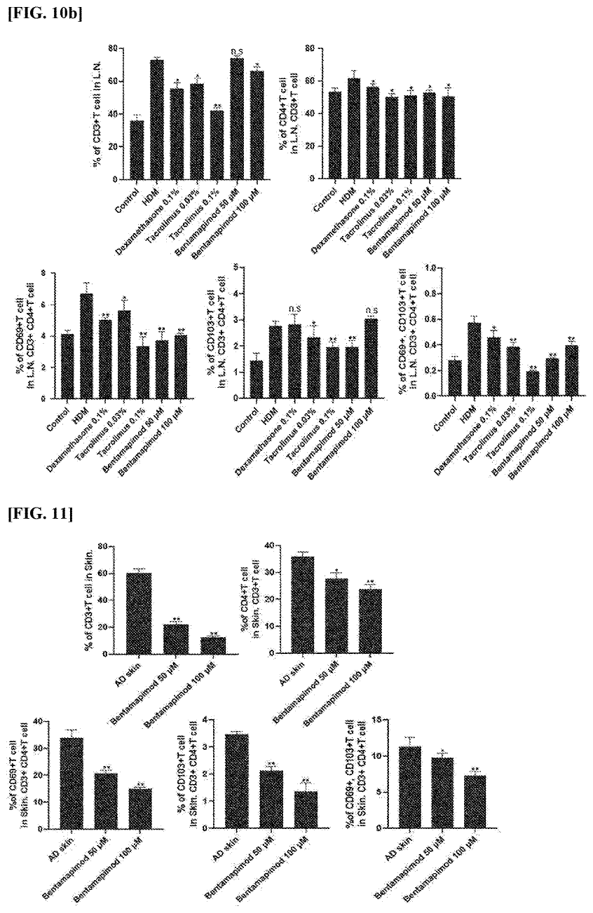

CD3+ T cells were also examined by FACS in skin-draining lymph nodes and found to be statistically significantly lower in the 100 μM bentamapimod group. Among CD3+ CD4+ T cells, the fraction of T cells expressing CD69 was also reduced, and furthermore, CD3+ CD4+ CD69+ CD103+ T cells were also statistically significantly lower in the 100 μM bentamapimod group, confirming a statistically significant reduction in the number of CD69+ T cells and CD103+ T cells, which are known to be increased in atopic dermatitis skin lesions. Furthermore, the percentage of T cells was significantly reduced compared to the 0.1% dexamethasone group used as a control, confirming that the composition of the present invention effectively treats the symptoms of atopic dermatitis through a multifaceted mechanism and controls immune and inflammatory responses in a skin tissue-specific manner, particularly by inhibiting skin-specific T cells (see FIGS. 10a and 10b). These results suggest that bentamapimod may have similar efficacy to topical steroids as a treatment for atopic dermatitis.

### 6.2 Identification of Changes in Skin-Specific T Cells in Human Atopic Dermatitis Lesion Skin

To test the effect of the candidate drug on skin-specific T cells in human skin tissue, a single suspension was prepared using skin from a patient with atopic dermatitis. T cells were stained using anti-CD3, anti-CD4, anti-CD8, and anti-CD45 antibodies, and flow cytometry was performed on skin T cells using the skin TRM markers CD69 and CD103 to observe changes in the number and distribution of skin-specific T cells in response to drug application.

In our experiments, we examined immune cell fractions derived from skin tissue of atopic dermatitis patients by FACS and found a significant dose-dependent decrease in the percentage of skin-specific T cells in both groups at 50 μM and 100 μM of bentamapimod (see FIG. 11).

Taken together, the results demonstrate that the compositions of the present invention effectively treat the symptoms of atopic dermatitis while ameliorating the side effects of conventional drugs through multiple mechanisms, and specifically control immune and inflammatory responses in a skin tissue-specific manner by inhibiting skin-specific T cells.

While the foregoing has described certain aspects of the present invention in detail, it will be apparent to one of ordinary skill in the art that these specific descriptions are merely preferred embodiments and are not intended to limit the scope of the invention. Accordingly, it will be understood that the substantial scope of the invention is defined by the appended claims and their equivalents.

### INDUSTRIAL APPLICABILITY

By specifically inhibiting the activity of skin-specific T cells, the compositions of the present invention can efficiently ameliorate inflammation-induced skin tissue damage while minimizing the impact on systemic immune activity, and thus can be used as a preventive or therapeutic agent for inflammatory or autoimmune skin diseases.

1. A method for preventing or treating inflammatory or autoimmune skin disease, comprising administering to a subject in need thereof a pharmaceutical composition comprising a compound of the following Formula 1 or a pharmaceutically acceptable salt thereof as an active ingredient:

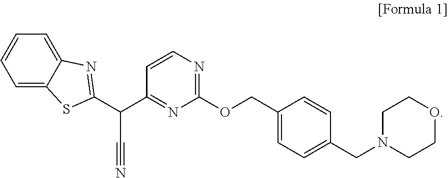 [SMILES: N#CC(c1ccnc(OCc2ccc(CN3CCOCC3)cc2)n1)c1nc2ccccc2s1]

2. The method according to claim 1, wherein the composition inhibits the proliferation of T cells.

3. The method according to claim 2, wherein the T cells are skin-specific T cells.

4. The method according to claim 1, wherein the inflammatory or autoimmune skin disease is selected from the group consisting of atopic dermatitis, eczema, psoriasis, allergy, erythema multiforme, erythema nodosum, and pyoderma gangrenosum.

5. The method according to claim 4, wherein the inflammatory or autoimmune skin disease is atopic dermatitis.

6. A cosmetic method for preventing or ameliorating inflammatory or autoimmune skin disease, comprising applying to a subject in need thereof a cosmetic composition comprising a compound of the following Formula 1 or a cosmetically acceptable salt thereof as an active ingredient:

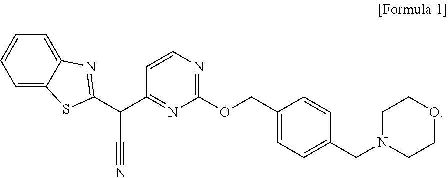 [SMILES: N#CC(c1ccnc(OCc2ccc(CN3CCOCC3)cc2)n1)c1nc2ccccc2s1]

7. The method according to claim 6, wherein the composition inhibits the proliferation of T cells.

8. The method according to claim 7, wherein the T cells are skin-specific T cells.

9. The method according to claim 6, wherein the inflammatory or autoimmune skin disease is selected from the group consisting of atopic dermatitis, eczema, psoriasis, allergy, erythema multiforme, erythema nodosum, and pyoderma gangrenosum.

10. The method according to claim 9, wherein the inflammatory or autoimmune skin disease is atopic dermatitis.

11. The method according to claim 1, wherein the method comprises administering transdermal the pharmaceutical composition comprising the compound of the Formula 1 or a pharmaceutically acceptable salt thereof as an active ingredient.

12. The method according to claim 1, wherein the method comprises administering topically to the skin the pharmaceutical composition comprising the compound of the Formula 1 or a pharmaceutically acceptable salt thereof as an active ingredient.

13. A method for screening a composition for inhibiting skin-specific T cells, comprising steps of:
(a) bringing a candidate substance into contact with a biological sample containing cells expressing an c-Jun N-terminal kinases (JNK) protein; and
(b) measuring the activity or expression level of the JNK protein in the sample,
wherein, if the activity or expression level of the JNK protein decreased, the candidate substance is determined as the composition for inhibiting skin-specific T cells.

14. The method according to claim 11, wherein the biological sample contains skin tissue or skin tissue-derived cells.
